(19) United States
(12) Patent Application Publication
(10) Pub. No.: US 2018/0028141 A1
(43) Pub. Date: Feb. 1, 2018

(54) RADIOGRAPHY SYSTEM, RADIOGRAPHY METHOD, AND RADIOGRAPHY PROGRAM

(71) Applicant: FUJIFILM CORPORATION, Tokyo (JP)

(72) Inventor: Takeshi KUWABARA, Kanagawa (JP)

(21) Appl. No.: 15/648,462

(22) Filed: Jul. 13, 2017

(30) Foreign Application Priority Data
Jul. 29, 2016 (JP) 2016-150590

(51) Int. Cl. A61B 6/00

(52) U.S. Cl. CPC A61B 6/54; A61B 6/4266; A61B 6/4233; A61B 6/56

(57) ABSTRACT

A radiography system includes: a radiography apparatus including a first radiation detector and a second radiation detector which is provided on the side of the first radiation detector from which the radiation is transmitted and emitted; and an integrated control unit that controls a charge accumulation operation in the first radiation detector and a charge accumulation operation in the second radiation detector, on the basis of the detection result of the time related to the emission of the radiation by an electric signal which is obtained by converting charge generated in the pixels of the first radiation detector and of which the level increases as the amount of charge increases.

### CROSS-REFERENCE TO RELATED APPLICATIONS

The present application claims priority under 35 U.S.C § 119 to Japanese Patent Application No. 2016-150590, filed on Jul. 29, 2016, which is hereby expressly incorporated by reference, in its entirety, into the present application.

### BACKGROUND

### Technical Field

The present disclosure relates to a radiography system, a radiography method, and a radiography program.

### Related Art

For example, as disclosed in WO2013/047193A, a radiography apparatus has been known that includes two radiation detectors each of which includes a plural pixels that accumulate a larger amount of charge as they are irradiated with a larger amount of radiation and which are provided so as to be stacked.

In addition, a technique has been known which detects the time related to the emission of radiation, such as the time when the emission of radiation starts and the time when the emission of radiation ends, on the basis of an electric signal of which the level generally increases as the amount of charge output from each pixel of a radiation detector of a radiography apparatus increases and controls an operation related to the accumulation of charge in each pixel.

However, in a case in which radiographic images are captured by two radiation detectors disclosed in, for example, WO2013/047193A, radiation that has been transmitted through the radiation detector provided on the incident side of the radiation reaches the radiation detector provided on the emission side of the radiation. Therefore, the amount of radiation that reaches the radiation detector provided on the emission side of the radiation is less than the amount of radiation that reaches the radiation detector provided on the incident side and the amount of radiation used to generate a radiographic image is reduced.

Therefore, in the radiation detector provided on the incident side of the radiation and the radiation detector provided on the emission side of the radiation, the detection results of the time related to the emission of radiation are different from each other. As a result, in some cases, the accumulation of charge in each pixel of each radiation detector is asynchronous.

### SUMMARY

The present disclosure has been made in view of the above-mentioned problems and an object of the present disclosure is to provide a technique that can synchronize the accumulation of charge even when the amount of radiation emitted to a second radiation detector is less than the amount of radiation emitted to a first radiation detector.

In order to achieve the object, according to an aspect of the invention, there is provided a radiography system including: a radiography apparatus including a first radiation detector in which a first plural pixels, each of which includes a conversion element that generates a larger amount of charge as it is irradiated with a larger amount of radiation, are two-dimensionally arranged, and a second radiation detector provided on a side of the first radiation detector from which the radiation is transmitted and emitted and in which a second plural pixels, each of which includes a conversion element that generates a larger amount of charge as it is irradiated with a larger amount of radiation, are two-dimensionally arranged; and a controller that executes a process, the process including: obtaining an electric signal, which is converted from charge generated in the first plural pixels and of which the level increases as the amount of charge increases; detecting a time related to the emission of the radiation from the obtained electric signal; and controlling a first charge accumulation operation in the first plural pixels and a second charge accumulation operation in the second plural pixels on the basis of the detected time.

In order to achieve the object, according to another aspect of the present disclosure, there is provided a radiography method that is performed by a radiography apparatus including a first radiation detector in which a first plural pixels, each of which includes a conversion element that generates a larger amount of charge as it is irradiated with a larger amount of radiation, are two-dimensionally arranged, and a second radiation detector which is provided on a side of the first radiation detector from which the radiation is transmitted and emitted and in which a first plural pixels, each of which includes a conversion element that generates a larger amount of charge as it is irradiated with a larger amount of radiation, are two-dimensionally arranged, the method including: obtaining an electric signal, which is converted from charge generated in the first plural pixels and of which the level increases as the amount of charge increases; detecting a time related to the emission of the radiation from the obtained electric signal; and controlling a first charge accumulation operation in the first plural pixels and a second charge accumulation operation in the first plural pixels on the basis of the detected time.

In order to achieve the object, according to still another aspect of the present disclosure, there is provided A non-transitory computer readable storage medium storing a radiography program that causes a computer to execute a process of controlling a radiography apparatus, the radiography apparatus including: a first radiation detector in which a first plural pixels, each of which includes a conversion element that generates a larger amount of charge as it is irradiated with a larger amount of radiation, are two-dimensionally arranged, and a second radiation detector which is provided on a side of the first radiation detector from which the radiation is transmitted and emitted and in which a first plural pixels, each of which includes a conversion element that generates a larger amount of charge as it is irradiated with a larger amount of radiation, are two-dimensionally arranged, and the process including: obtaining an electric signal, which is converted from charge generated in the first plural pixels and of which the level increases as the amount of charge increases; detecting a time related to the emission of the radiation from the obtained electric signal; and controlling a first charge accumulation operation in the first plural pixels and a second charge accumulation operation in the first plural pixels on the basis of the detected time.

### BRIEF DESCRIPTION OF THE DRAWINGS

FIG. 1 is a block diagram illustrating an example of the structure of a radiography system according to an embodiment.

FIG. 2 is a side cross-sectional view illustrating an example of the structure of a radiography apparatus according to this embodiment.

FIG. 3 is a block diagram illustrating an example of the structure of a main portion of an electric system of the radiography apparatus according to this embodiment.

FIG. 4 is a circuit diagram illustrating an example of the structure of a signal processing unit according to this embodiment.

FIG. 5 is a block diagram illustrating an example of the structure of a main portion of an electric system of a console according to this embodiment.

FIG. 6 is a graph illustrating the amount of radiation that reaches each of a first radiation detector and a second radiation detector according to this embodiment.

FIG. 7 is a flowchart illustrating an example of the flow of an overall imaging process according to this embodiment.

FIG. 8 is a flowchart illustrating an example of the flow of an image generation process in the overall imaging process according to this embodiment.

FIG. 9 is a front view schematically illustrating a bone tissue region and a soft tissue region according to this embodiment.

FIG. 10 is a timing chart illustrating an example of the flow of the capture of a radiographic image by the radiography apparatus 16 according to this embodiment.

FIG. 11 is a diagram schematically illustrating a change in the amount of radiation emitted from a radiation source over an irradiation time.

FIG. 12 is a flowchart illustrating an example of the flow of an accumulation synchronization process according to this embodiment.

FIG. 13 is a flowchart illustrating an example of the flow of a first imaging process according to this embodiment.

FIG. 14 is a flowchart illustrating an example of the flow of a second imaging process according to this embodiment.

FIG. 15 is a timing chart illustrating another example of oversampling in the second radiation detector according to this embodiment.

FIG. 16 is a timing chart illustrating still another example of the oversampling in the second radiation detector according to this embodiment.

FIG. 17 is a timing chart illustrating an example of a reading method for collectively reading charge from pixels connected to a plurality of adjacent gate lines.

FIG. 18 is a timing chart illustrating an example of a reading method for collectively reading charge from pixels connected to a plurality of adjacent data lines.

### DETAILED DESCRIPTION

Hereinafter, an embodiment of the invention will be described in detail with reference to the drawings.

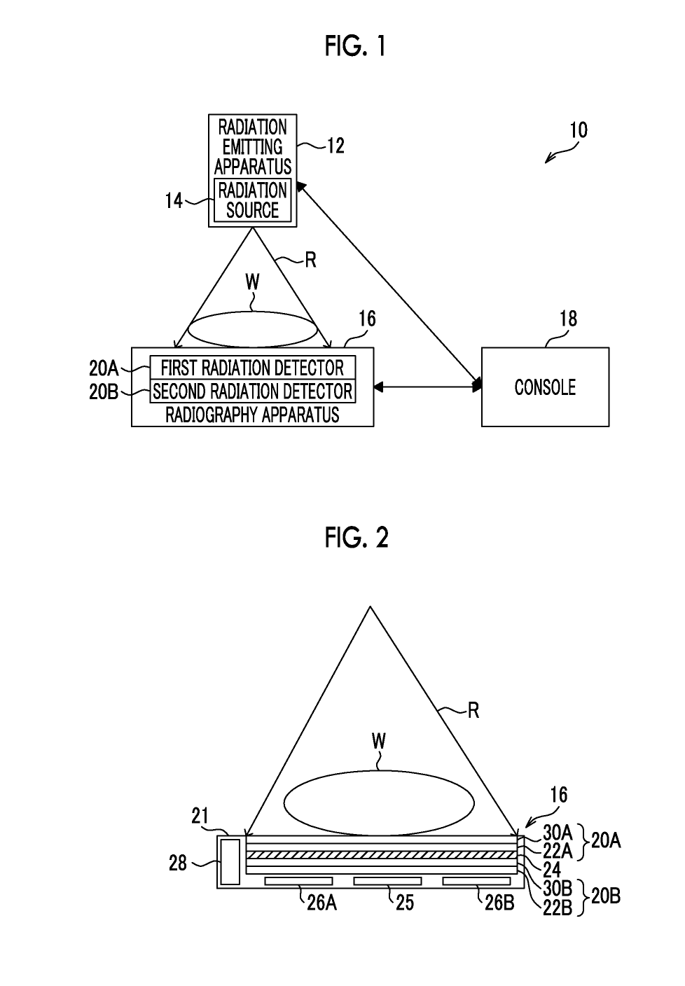

First, the structure of a radiography system 10 according to this embodiment will be described with reference to FIG. 1. As illustrated in FIG. 1, the radiography system 10 includes a radiation emitting apparatus 12, a radiography apparatus 16, and a console 18. The console 18 according to this embodiment is an example of an image processing apparatus according to the invention.

The radiation emitting apparatus 12 according to this embodiment includes a radiation source 14 that irradiates a subject W, which is an example of an imaging target, with radiation R such as X-rays. An example of the radiation emitting apparatus 12 is a treatment cart. A method for instructing the radiation emitting apparatus 12 to emit the radiation R is not particularly limited. For example, in a case in which the radiation emitting apparatus 12 includes an irradiation button, a user, such as a doctor or a radiology technician, may press the irradiation button to instruct the emission of the radiation R such that the radiation R is emitted from the radiation emitting apparatus 12. In addition, for example, the user may operate the console 18 to instruct the emission of the radiation R such that the radiation R is emitted from the radiation emitting apparatus 12.

When receiving a command to start the emission of the radiation R, the radiation emitting apparatus 12 emits the radiation R from the radiation source 14 according to exposure conditions, such as a tube voltage, a tube current, and an irradiation period.

The radiography apparatus 16 according to this embodiment includes a first radiation detector 20A and a second radiation detector 20B that detect the radiation R which has been emitted from the radiation emitting apparatus 12 and then transmitted through the subject W. The radiography apparatus 16 captures radiographic images of the subject W using the first radiation detector 20A and the second radiation detector 20B. Hereinafter, in a case in which the first radiation detector 20A and the second radiation detector 20B do not need to be distinguished from each other, they are generically referred to as “radiation detectors 20”.

Next, the structure of the radiography apparatus 16 according to this embodiment will be described with reference to FIG. 2. As illustrated in FIG. 2, the radiography apparatus 16 includes a plate-shaped housing 21 that transmits the radiation R and has a waterproof, antibacterial, and airtight structure. The housing 21 includes the first radiation detector 20A, the second radiation detector 20B, a radiation limitation member 24, a control board 25, a control board 26A, a control board 26B, and a case 28.

The first radiation detector 20A is provided on the incident side of the radiation R and the second radiation detector 20B is provided so as to be stacked on the side of the first radiation detector 20A from which the radiation R is transmitted and emitted in the radiography apparatus 16. The first radiation detector 20A includes a thin film transistor (TFT) substrate 30A and a scintillator 22A which is an example of a light emitting layer that is irradiated with the radiation R and emits light corresponding to the amount of radiation R emitted. The TFT substrate 30A and the scintillator 22A are stacked in the order of the TFT substrate 30A and the scintillator 22A from the incident side of the radiation R. The term “stacked” means a state in which the first radiation detector 20A and the second radiation detector 20B overlap each other in a case in which the first radiation detector 20A and the second radiation detector 20B are seen from the incident side or the emission side of the radiation R in the radiography apparatus 16 and it does not matter how they overlap each other. For example, the first radiation detector 20A and the second radiation detector 20B, or the first radiation detector 20A, the radiation limitation member 24, and the second radiation detector 20B may overlap while coming into contact with each other or may overlap with a gap therebetween in the stacking direction.

The second radiation detector 20B includes a TFT substrate 30B and a scintillator 22B which is an example of the light emitting layer. The TFT substrate 30B and the scintillator 22B are stacked in the order of the TFT substrate 30B and the scintillator 22B from the incident side of the radiation R.

That is, the first radiation detector 20A and the second radiation detector 20B are so-called irradiation side sampling (ISS) radiation detectors that are irradiated with the radiation R from the side of the TFT substrates 30A and 30B.

In the radiography apparatus 16 according to this embodiment, the scintillator 22A of the first radiation detector 20A and the scintillator 22B of the second radiation detector 20B have different compositions. Specifically, for example, the scintillator 22A includes CsI (Tl) (cesium iodide having thallium added thereto) as a main component and the scintillator 22B includes gadolinium oxysulfide (GOS) as a main component. GOS has a higher sensitivity to the high-energy radiation R than CsI. In addition, a combination of the composition of the scintillator 22A and the composition of the scintillator 22B is not limited to the above-mentioned example and may be a combination of other compositions or a combination of the same compositions.

The radiation limitation member 24 that limits the transmission of the radiation R is provided between the first radiation detector 20A and the second radiation detector 20B. An example of the radiation limitation member 24 is a metal plate made of, for example, copper or tin. It is preferable that a variation in the thickness of the radiation limitation member 24 in the incident direction of the radiation R is equal to or less than 1% in order to uniformize limitations (transmissivity) on the radiation.

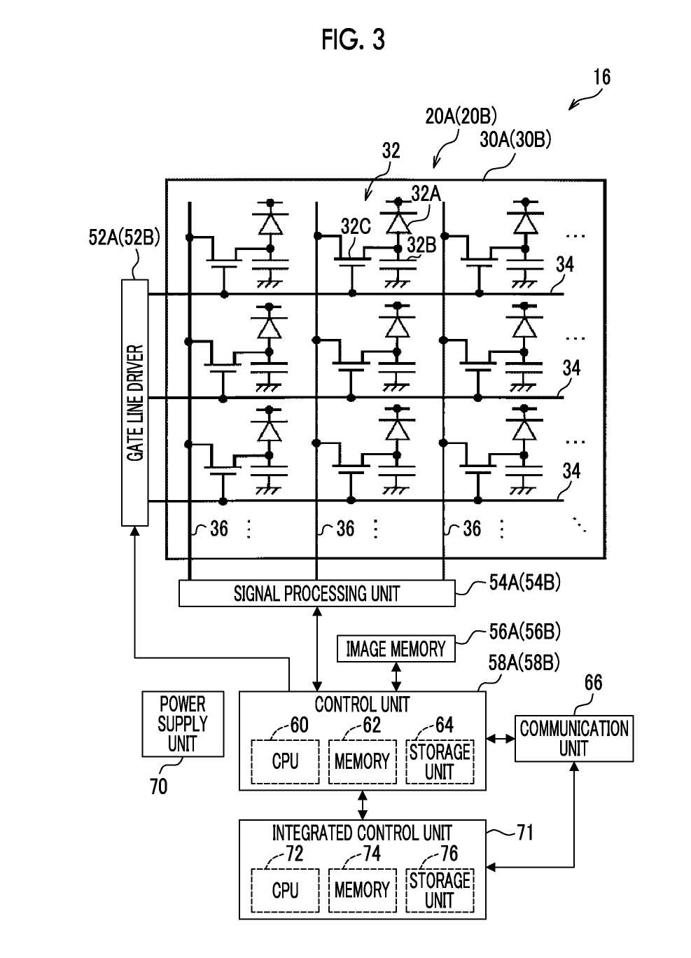

An electronic circuit, such as an integrated control unit 71 (see FIG. 3) which will be described below, is formed on the control board 25. The control board 26A is provided so as to correspond to the first radiation detector 20A and electronic circuits, such as an image memory 56A and a control unit 58A which will be described below, are formed on the control board 26A. The control board 26B is provided so as to correspond to the second radiation detector 20B and electronic circuits, such as an image memory 56B and a control unit 58B which will be described below, are formed on the control board 26B. The control board 25, the control board 26A, and the control board 26B are provided on the side of the second radiation detector 20B which is opposite to the incident side of the radiation R.

As illustrated in FIG. 2, the case 28 is provided at a position (that is, outside the range of an imaging region) that does not overlap the radiation detector 20 at one end of the housing 21. For example, a power supply unit 70 which will be described below is accommodated in the case 28. The installation position of the case 28 is not particularly limited. For example, the case 28 may be provided at a position that overlaps the radiation detector 20 on the side of the second radiation detector 20B which is opposite to the incident side of the radiation R.

Next, the structure of a main portion of an electric system of the radiography apparatus 16 according to this embodiment will be described with reference to FIG. 3.

As illustrated in FIG. 3, a plurality of pixels 32 are two-dimensionally provided in one direction (a row direction in FIG. 3) and an intersection direction (a column direction in FIG. 3) that intersects the one direction on the TFT substrate 30A. The pixel 32 includes a sensor unit 32A, a capacitor 32B, and a field effect thin film transistor (TFT; hereinafter, simply referred to as a “thin film transistor”) 32C. The sensor unit 32A according to this embodiment is an example of a conversion element according to the invention.

The sensor unit 32A includes, for example, an upper electrode, a lower electrode, and a photoelectric conversion film which are not illustrated, absorbs the light emitted from the scintillator 22A, and generates charge. The capacitor 32B accumulates the charge generated by the sensor unit 32A. The thin film transistor 32C reads the charge accumulated in the capacitor 32B and outputs the charge in response to a control signal. The charge, of which the amount increases as the amount of radiation emitted increases, is accumulated in the pixel 32 according to this embodiment by the above-mentioned structure.

A plurality of gate lines 34 which extend in the one direction and are used to turn on and off each thin film transistor 32C are provided on the TFT substrate 30A. In addition, a plurality of data lines 36 which extend in the intersection direction and to which the charge read by the thin film transistors 32C in an on state is output are provided on the TFT substrate 30A.

A gate line driver 52A is provided on one side of two adjacent sides of the TFT substrate 30A and a signal processing unit 54A is provided on the other side. Each gate line 34 of the TFT substrate 30A is connected to the gate line driver 52A and each data line 36 of the TFT substrate 30A is connected to the signal processing unit 54A.

The thin film transistors 32C corresponding to each gate line 34 on the TFT substrate 30A are sequentially turned on (in units of row illustrated in FIG. 3 in this embodiment) by control signals which are supplied from the gate line driver 52A through the gate lines 34. The charge which is read by the thin film transistor 32C in an on state is transmitted as an electric signal through the data line 36 and is input to the signal processing unit 54A. In this way, charge is sequentially read from each gate line 34 (in units of row illustrated in FIG. 3 in this embodiment) and image data indicating a two-dimensional radiographic image is generated by the signal processing unit 54A.

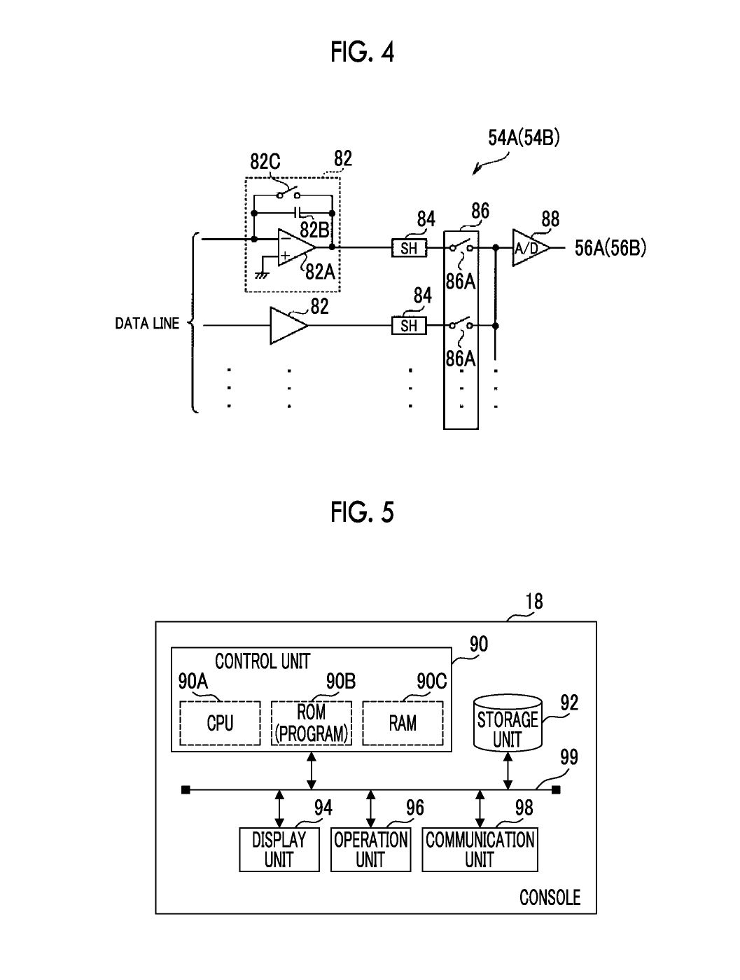

As illustrated in FIG. 4, the signal processing unit 54A includes a variable gain pre-amplifier (charge amplifier) 82 and a sample-and-hold circuit 84 which correspond to each data line 36.

The variable gain pre-amplifier 82 includes an operational amplifier 82A that has a positive input side grounded and a capacitor 82B and a reset switch 82C that are connected in parallel to each other between a negative input side and an output side of the operational amplifier 82A. The reset switch 82C is turned on and off by the control unit 58A. The variable gain pre-amplifier 82 according to this embodiment is an example of an amplifier according to the invention.

In addition, the signal processing unit 54A according to this embodiment includes a multiplexer 86 and an analog/digital (A/D) converter 88. The sampling time of the sample-and-hold circuit 84 and the turn-on and turn-off of a switch 86A provided in the multiplexer 86 are controlled by the control unit 58A.

When a radiographic image is detected, first, the control unit 58A maintains the reset switch 82C of the variable gain pre-amplifier 82 in an on state for a predetermined period to release the charge accumulated in the capacitor 82B.

When the connected thin film transistor 32C is turned on, the charge that is accumulated in the capacitor 32B of each pixel 32 irradiated with the radiation R is transmitted as an electric signal through the connected data line 36. The electric signal transmitted through the data line 36 is amplified at a predetermined gain by the corresponding variable gain pre-amplifier 82.

After the above-mentioned discharging is performed, the control unit 58A drives the sample-and-hold circuit 84 for a predetermined period such that the level of the electric signal amplified by the variable gain pre-amplifier 82 is held and sampled by the sample-and-hold circuit 84.

Then, the signal levels sampled by each sample-and-hold circuit 84 are sequentially selected by the multiplexer 86 and are then converted into digital signal levels by the A/D converter 88 under the control of the control unit 58A. In this way, image data indicating the captured radiographic image is acquired.

The signal processing unit 54B of the second radiation detector 20B and the signal processing unit 54A of the first radiation detector 20A have the same structure except that the gains of the variable gain pre-amplifiers 82 are different from each other. The description of the same structure will not be repeated here.

In the radiography apparatus 16 according to this embodiment, since the first radiation detector 20A and the radiation limitation member 24 absorb the radiation R, the amount of radiation that reaches the second radiation detector 20B is less than the amount of radiation that reaches the first radiation detector 20A. Therefore, the amount of charge generated in each pixel 32 of the second radiation detector 20B is less than the amount of charge generated in each pixel 32 of the first radiation detector 20A.

Therefore, in the radiography apparatus 16 according to this embodiment, the gain of the variable gain pre-amplifier 82 in the signal processing unit 54B of the second radiation detector 20B is higher than the gain of the variable gain pre-amplifier 82 in the signal processing unit 54A of the first radiation detector 20A. The amount of radiation R that is absorbed before reaching the second radiation detector 20B varies depending on, for example, the material forming the radiation limitation member 24. In a case in which the gain of the variable gain pre-amplifier 82 is too high, the capacitor 82B is likely to be saturated. Therefore, specifically, the gain of the variable gain pre-amplifier 82 may be a value that is obtained in advance by, for example, experiments and is in the range in which the capacitor 82B is not saturated. For example, it is preferable that the gain of the variable gain pre-amplifier 82 in the second radiation detector 20B is 2 to 10 times higher than the gain of the variable gain pre-amplifier 82 in the first radiation detector 20A, considering, for example, the material forming the radiation limitation member 24.

A method for setting the gain of the variable gain pre-amplifier 82 in the second radiation detector 20B to be higher than the gain of the variable gain pre-amplifier 82 in the first radiation detector 20A is not particularly limited. For example, since the gain of the variable gain pre-amplifier 82 increases as the capacitance of the capacitor 82B increases, the capacitance of the capacitor 82B in the variable gain pre-amplifier 82 of the second radiation detector 20B may be higher than that in the first radiation detector 20A. In addition, the gain of the variable gain pre-amplifier 82 in the second radiation detector 20B may be variable. For example, a plurality of series circuits, each of which includes a switch and a capacitor, may be connected in parallel to the capacitor 82B (operational amplifier 82A) and the switches may be turned on and off to change the number of capacitors connected to the operational amplifier 82A. In this way, the gain is changed.

The gain of the variable gain pre-amplifier 82 in the second radiation detector 20B in a case in which the signal processing unit 54B generates image data indicating the radiographic image captured by the second radiation detector 20B may be higher than the gain of the variable gain pre-amplifier 82 in the first radiation detector 20A in a case in which the signal processing unit 54A generates image data indicating the radiographic image captured by the first radiation detector 20A. In other cases, the gain is not particularly limited.

The image memory 56A is connected to the signal processing unit 54A through the control unit 58A. The image data output from the A/D converter 88 of the signal processing unit 54A is sequentially output to the control unit 58A. The image memory 56A is connected to the control unit 58A. The image data sequentially output from the signal processing unit 54A is sequentially stored in the image memory 56A under the control of the control unit 58A. The image memory 56A has memory capacity that can store a predetermined amount of image data. Whenever a radiographic image is captured, captured image data is sequentially stored in the image memory 56A. In addition, the image memory 56A is connected to the control unit 58A.

The control unit 58A includes a central processing unit (CPU) 60, a memory 62 including, for example, a read only memory (ROM) and a random access memory (RAM), and a non-volatile storage unit 64 such as a flash memory. An example of the control unit 58A is a microcomputer.

The integrated control unit 71 includes a CPU 72, a memory 74 including, for example, a ROM and a RAM, and a non-volatile storage unit 76 such as a flash memory. An example of the integrated control unit 71 is a microcomputer. The control unit 58A and the integrated control unit 71 are connected such that they can communicate with each other.

The integrated control unit 71 according to this embodiment has a function that determines whether the emission of the radiation R has started on the basis of whether the value of the digital signal output from the control unit 58A is equal to or greater than a predetermined threshold value and controls the control unit 58A and the control unit 58B such that the control unit 58A and the control unit 58B control an operation of accumulating charge in each pixel 32 and start the accumulation of the charge in a case in which it is determined that the emission of the radiation R has started, which will be described in detail below.

A communication unit 66 is connected to the control unit 58A and the integrated control unit 71 and transmits and receives various kinds of information to and from external apparatuses, such as the radiation emitting apparatus 12 and the console 18, using at least one of wireless communication or wired communication. The power supply unit 70 supplies power to each of the above-mentioned various circuits or elements (for example, the gate line driver 52A, the signal processing unit 54A, the image memory 56A, the control unit 58A, the communication unit 66, and the integrated control unit 71). In FIG. 3, lines for connecting the power supply unit 70 to various circuits or elements are not illustrated in order to avoid complication.

Components of the TFT substrate 30B, the gate line driver 52B, the signal processing unit 54B, the image memory 56B, and the control unit 58B of the second radiation detector 20B have the same structures as the corresponding components of the first radiation detector 20A and thus the description thereof will not be repeated here. The control unit 58A and the control unit 58B are connected such that they can communicate with each other.

According to the above-mentioned structure, the radiography apparatus 16 according to this embodiment captures radiographic images using the first radiation detector 20A and the second radiation detector 20B.

Next, the structure of the console 18 according to this embodiment will be described with reference to FIG. 5. As illustrated in FIG. 5, the console 18 includes a control unit 90. The control unit 90 includes a CPU 90A that controls the overall operation of the console 18, a ROM 90B in which, for example, various programs or various parameters are stored in advance, and a RAM 90C that is used as, for example, a work area when the CPU 90A executes various programs.

In addition, the console 18 includes a non-volatile storage unit 92 such as a hard disk drive (HDD). The storage unit 92 stores and holds image data indicating a radiographic image captured by the first radiation detector 20A, image data indicating a radiographic image captured by the second radiation detector 20B, and various other data. Hereinafter, the radiographic image captured by the first radiation detector 20A is referred to as a “first radiographic image” and image data indicating the first radiographic image is referred to as “first radiographic image data”. In addition, hereinafter, the radiographic image captured by the second radiation detector 20B is referred to as a “second radiographic image” and image data indicating the second radiographic image is referred to as “second radiographic image data”. In a case in which the “first radiographic image” and the “second radiographic image” are generically named, they are simply referred to as “radiographic images”.

The console 18 further includes a display unit 94, an operation unit 96, and a communication unit 98. The display unit 94 displays, for example, information related to imaging and a captured radiographic image. The user uses the operation unit 96 to input, for example, a command to capture a radiographic image and a command related to image processing for a captured radiographic image. For example, the operation unit 96 may have the form of a keyboard or may have the form of a touch panel that is integrated with the display unit 94. The communication unit 98 transmits and receives various kinds of information to and from the radiation emitting apparatus 12 and the radiography apparatus 16, using at least one of wireless communication or wired communication. In addition, the communication unit 98 transmits and receives various kinds of information to and from external systems, such as a picture archiving and communication system (PACS) and a radiology information system (RIS), using at least one of wireless communication or wired communication.

The control unit 90, the storage unit 92, the display unit 94, the operation unit 96, and the communication unit 98 are connected to each other through a bus 99.

As described above, in the radiography apparatus 16 according to this embodiment, the amount of radiation that reaches the second radiation detector 20B is less than the amount of radiation that reaches the first radiation detector 20A. In addition, the radiation limitation member 24 generally has the characteristic that it absorbs a larger number of low-energy components than high-energy components in energy forming the radiation R, which depends on the material forming the radiation limitation member 24. Therefore, the energy distribution of the radiation R that reaches the second radiation detector 20B has a larger number of high-energy components than the energy distribution of the radiation R that reaches the first radiation detector 20A.

In this embodiment, for example, about 50% of the radiation R that has reached the first radiation detector 20A is absorbed by the first radiation detector 20A and is used to capture a radiographic image. In addition, about 60% of the radiation R that has passed through the first radiation detector 20A and reached the radiation limitation member 24 is absorbed by the radiation limitation member 24. About 50% of the radiation R that has passed through the first radiation detector 20A and the radiation limitation member 24 and reached the second radiation detector 20B is absorbed by the second radiation detector 20B and is used to capture a radiographic image.

That is, the amount of radiation (the amount of charge generated by the second radiation detector 20B) used to capture a radiographic image by the second radiation detector 20B is about 20% of the amount of radiation used to capture a radiographic image by the first radiation detector 20A. In addition, the ratio of the amount of radiation used to capture a radiographic image by the second radiation detector 20B to the amount of radiation used to capture a radiographic image by the first radiation detector 20A is not limited to the above-mentioned ratio. However, it is preferable that the amount of radiation used to capture a radiographic image by the second radiation detector 20B is equal to or greater than 10% of the amount of radiation used to capture a radiographic image by the first radiation detector 20A in terms of diagnosis.

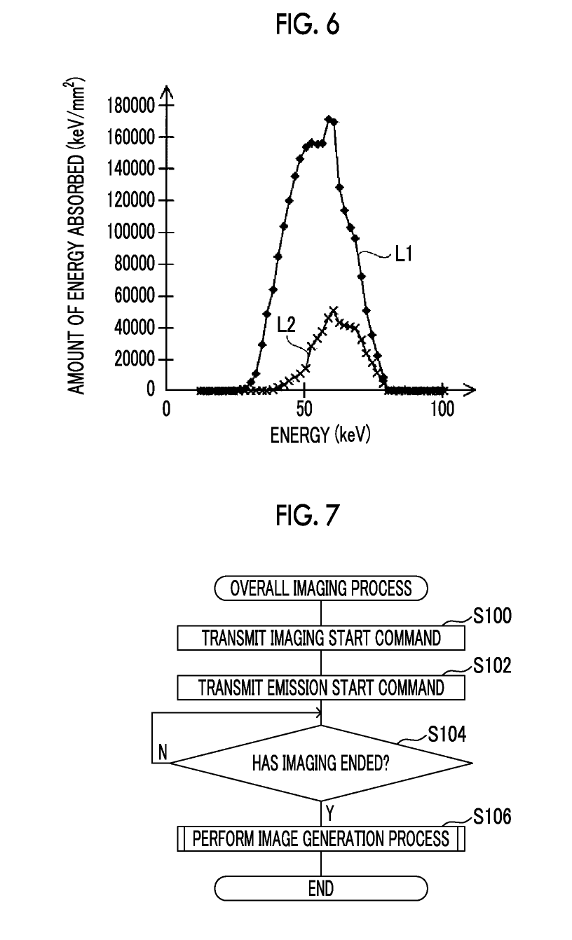

The radiation R is absorbed from a low-energy component. Therefore, for example, as illustrated in FIG. 6, the energy components of the radiation R that reaches the second radiation detector 20B do not include the low-energy components of the energy components of the radiation R that reaches the first radiation detector 20A. In FIG. 6, the vertical axis indicates the amount of radiation R absorbed per unit area and the horizontal axis indicates the energy of the radiation R in a case in which the tube voltage of the radiation source 14 is 80 kV. In addition, in FIG. 6, a solid line L1 indicates the relationship between the energy of the radiation R absorbed by the first radiation detector 20A and the amount of radiation R absorbed per unit area. In FIG. 6, a solid line L2 indicates the relationship between the energy of the radiation R absorbed by the second radiation detector 20B and the amount of radiation R absorbed per unit area.

Next, the operation of the radiography system 10 according to this embodiment will be described.

First, the operation of the console 18 will be described. FIG. 7 is a flowchart illustrating an example of the flow of an overall imaging process performed by the control unit 90 of the console 18. Specifically, the CPU 90A of the control unit 90 executes an overall imaging processing program to perform the overall imaging process illustrated in FIG. 7. The control unit 90 executes the overall imaging processing program to function as an example of a derivation unit according to the invention.

In this embodiment, the overall imaging process illustrated in FIG. 7 is performed in a case in which the control unit 90 of the console 18 acquires an imaging menu including, for example, the name of the subject W, an imaging part, and the emission conditions of the radiation R from the user through the operation unit 96. The control unit 90 may acquire the imaging menu from an external system, such as an RIS, or may acquire the imaging menu input by the user through the operation unit 96.

In Step S100 of FIG. 7, the control unit 90 of the console 18 transmits information included in the imaging menu as an imaging start command to the radiography apparatus 16 through the communication unit 98 and transmits the emission conditions of the radiation R to the radiation emitting apparatus 12 through the communication unit 98.

Then, in Step S102, the control unit 90 transmits a command to start the emission of the radiation R to the radiation emitting apparatus 12 through the communication unit 98. When receiving the emission conditions and the emission start command transmitted from the console 18, the radiation emitting apparatus 12 starts the emission of the radiation R according to the received emission conditions. The radiation emitting apparatus 12 may include an irradiation button. In this case, the radiation emitting apparatus 12 receives the emission conditions and the emission start command transmitted from the console 18 and starts the emission of the radiation R according to the received emission conditions in a case in which the irradiation button is pressed.

In the radiography apparatus 16, the first radiation detector 20A captures the first radiographic image and the second radiation detector 20B captures the second radiographic image, on the basis of the information in the imaging menu transmitted from the console 18, in response to the imaging start command, which will be described in detail below. In the radiography apparatus 16, the control units 58A and 58B perform various correction processes, such as offset correction and gain correction, for first radiographic image data indicating the captured first radiographic image and second radiographic image data indicating the captured second radiographic image, respectively, and store the corrected radiographic image data in the storage unit 64.

Then, in Step S104, the control unit 90 determines whether the capture of the radiographic images has ended in the radiography apparatus 16. A method for determining whether the capture of the radiographic images has ended is not particularly limited. For example, each of the control units 58A and 58B of the radiography apparatus 16 transmits end information indicating that imaging has ended to the console 18 through the communication unit 66. In a case in which the end information is received, the control unit 90 of the console 18 determines that the capture of the radiographic images has ended in the radiography apparatus 16.

For example, each of the control units 58A and 58B transmits the first radiographic image data and the second radiographic image data to the console 18 through the communication unit 66 after imaging ends. In a case in which the first radiographic image data and the second radiographic image data are received, the control unit 90 determines that the capture of the radiographic images by the radiography apparatus 16 has ended. In addition, in a case in which the first radiographic image data and the second radiographic image data are received, the console 18 stores the received first radiographic image data and the received second radiographic image data in the storage unit 92.

In a case in which the capture of the radiographic images by the radiography apparatus 16 has not ended, the determination result is “No” and the control unit 90 waits until the capture of the radiographic images by the radiography apparatus 16 ends. On the other hand, in a case in which the capture of the radiographic images by the radiography apparatus 16 has ended, the determination result is “Yes” and the control unit 90 proceeds to Step S106.

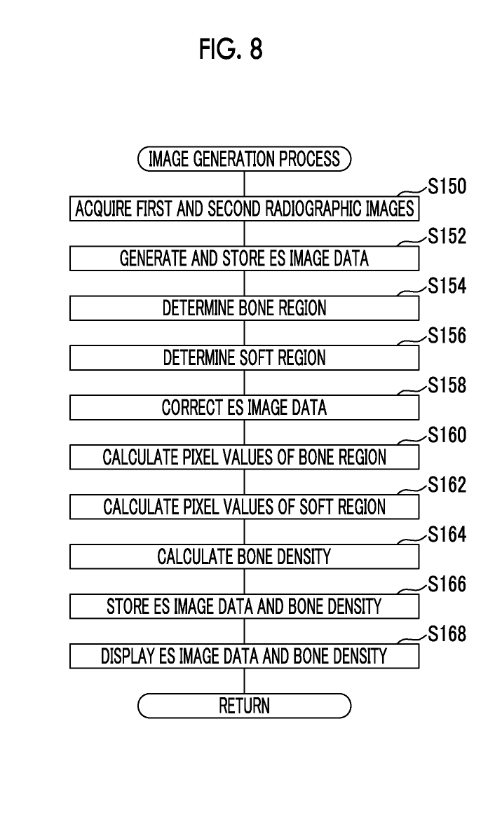

In Step S106, the control unit 90 performs an image generation process illustrated in FIG. 8 and ends the overall imaging process.

Next, the image generation process performed in Step S106 of the overall imaging process (see FIG. 7) will be described with reference to FIG. 8.

In Step S150 of FIG. 8, the control unit 90 of the console 18 acquires the first radiographic image data and the second radiographic image data. In a case in which the first radiographic image data and the second radiographic image data have been stored in the storage unit 92, the control unit 90 reads and acquires the first radiographic image data and the second radiographic image data from the storage unit 92. In a case in which the first radiographic image data and the second radiographic image data have not been stored in the storage unit 92, the control unit 90 acquires the first radiographic image data from the first radiation detector 20A and acquires the second radiographic image data from the second radiation detector 20B.

Then, in Step S152, the control unit 90 generates image data indicating an energy subtraction image, using the first radiographic image data and the second radiographic image data. Hereinafter, the energy subtraction image is referred to as an “ES image” and the image data indicating the energy subtraction image is referred to as “ES image data”.

In this embodiment, the control unit 90 subtracts image data obtained by multiplying the first radiographic image data by a predetermined coefficient from image data obtained by multiplying the second radiographic image data by a predetermined coefficient for each corresponding pixel. The control unit 90 generates ES image data indicating an ES image in which soft tissues have been removed and bone tissues have been highlighted, using the subtraction. A method for determining the corresponding pixels of the first radiographic image data and the second radiographic image data is not particularly limited. For example, the amount of positional deviation between the first radiographic image data and the second radiographic image data, which are captured by the radiography apparatus 16 in a state in which a marker is put in advance, may be calculated from the difference between the positions of the marker in the first radiographic image data and the second radiographic image data. Then, the corresponding pixels of the first radiographic image data and the second radiographic image data may be determined on the basis of the calculated amount of positional deviation.

In this case, for example, the amount of positional deviation between the first radiographic image data and the second radiographic image data, which are obtained by capturing the image of both the subject W and the marker when the image of the subject W is captured, may be calculated from the difference between the positions of the marker in the first radiographic image data and the second radiographic image data. In addition, for example, the amount of positional deviation between the first radiographic image data and the second radiographic image data may be calculated on the basis of the structure of the subject W in the first radiographic image data and the second radiographic image data obtained by capturing the image of the subject W.

Then, in Step S154, the control unit 90 determines a bone tissue region (hereinafter, referred to as a “bone region”) in the ES image that is indicated by the ES image data generated in Step S152. In this embodiment, for example, the control unit 90 estimates the approximate range of the bone region on the basis of the imaging part included in the imaging menu. Then, the control unit 90 detects pixels that are disposed in the vicinity of the pixels, of which the differential values are equal to or greater than a predetermined value, as the pixels forming the edge (end) of the bone region in the estimated range to determine the bone region.

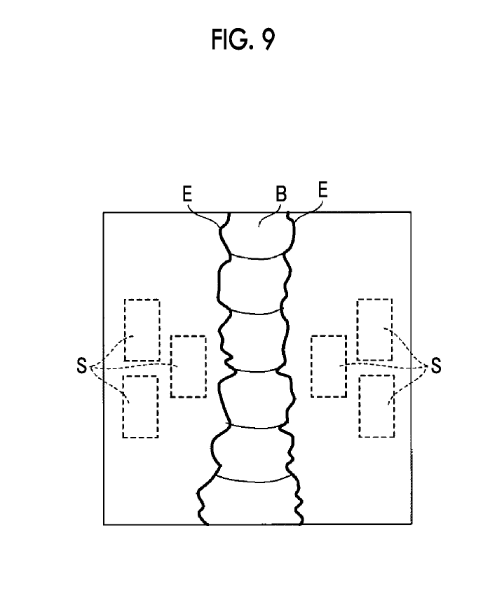

For example, as illustrated in FIG. 9, in Step S154, the control unit 90 detects the edge E of a bone region B and determines a region in the edge E as the bone region B. For example, FIG. 9 illustrates an ES image in a case in which the image of a backbone part of the upper half of the body of the subject W is captured.

A method for determining the bone region B is not limited to the above-mentioned example. For example, the control unit 90 displays the ES image that is indicated by the ES image data generated in Step S152 on the display unit 94. The user designates the edge E of the bone region B in the ES image displayed on the display unit 94 through the operation unit 96. Then, the control unit 90 may determine a region in the edge E designated by the user as the bone region B.

The control unit 90 may display an image in which the ES image and the edge E detected in Step S154 overlap each other on the display unit 94. In this case, in a case in which it is necessary to correct the edge E displayed on the display unit 94, the user corrects the position of the edge E through the operation unit 96. Then, the control unit 90 may determine a region in the edge E corrected by the user as the bone region B.

Then, in Step S156, the control unit 90 determines a soft tissue region (hereinafter, referred to as a “soft region”) in the ES image that is indicated by the ES image data generated in Step S152. In this embodiment, for example, the control unit 90 determines a region, which is other than the bone region B and has a predetermined area including pixels that are separated from the edge E by a distance corresponding to a predetermined number of pixels in a predetermined direction, as the soft region. For example, as illustrated in FIG. 9, in Step S156, the control unit 90 determines a plurality of (in the example illustrated in FIG. 9, six) soft regions S.

The predetermined direction and the predetermined number of pixels may be predetermined by, for example, experiments using the actual radiography apparatus 16 according to the imaging part. The predetermined area may be predetermined or may be designated by the user. In addition, for example, the control unit 90 may determine, as the soft region S, the pixels with pixel values in a predetermined range having the minimum pixel value (a pixel value corresponding to a position where the body thickness of the subject W is the maximum except the bone region B) as the lower limit in the ES image data. In addition, it goes without saying that the number of soft regions S determined in Step S156 is not limited to that illustrated in FIG. 9.

Then, in Step S158, the control unit 90 corrects the ES image data generated in Step S152 such that a variation in the ES image in each imaging operation is within an allowable range. In this embodiment, for example, the control unit 90 performs a correction process of removing image blur in the entire frequency band of the ES image data. The image data corrected in Step S158 is used to calculate bone density in a process from Step S160 to Step S164 which will be described below. Therefore, hereinafter, the corrected image data is referred to as “dual-energy X-ray absorptiometry (DXA) image data”.

Then, in Step S160, the control unit 90 calculates an average value A1 of the pixel values of the bone region B in the DXA image data. Then, in Step S162, the control unit 90 calculates an average value A2 of the pixel values of all of the soft regions S in the DXA image data. Here, in this embodiment, for example, the control unit 90 performs weighting such that the soft region S which is further away from the edge E has a smaller pixel value and calculates the average value A2. Before the average values A1 and A2 are calculated in Step S160 and Step S162, respectively, abnormal values of the pixel values of the bone region B and the pixel values of the soft region S may be removed by, for example, a median filter.

Then, in Step S164, the control unit 90 calculates the bone density of the imaging part of the subject W. In this embodiment, for example, the control unit 90 calculates the difference between the average value A1 calculated in Step S160 and the average value A2 calculated in Step S162. In addition, the control unit 90 multiplies the calculated difference by a conversion coefficient for converting the pixel value into bone mass [g] to calculate the bone mass. Then, the control unit 90 divides the calculated bone mass by the area [cm²] of the bone region B to calculate bone density [g/cm²]. The conversion coefficient may be predetermined by, for example, experiments using the actual radiography apparatus 16 according to the imaging part.

Then, in Step S166, the control unit 90 stores the ES image data generated in Step S152 and the bone density calculated in Step S164 in the storage unit 92 so as to be associated with information for identifying the subject W. In addition, for example, the control unit 90 may store the ES image data generated in Step S152, the bone density calculated in Step S164, the first radiographic image data, and the second radiographic image data in the storage unit 92 so as to be associated with the information for identifying the subject W.

Then, in Step S168, the control unit 90 displays the ES image indicated by the ES image data generated in Step S152 and the bone density calculated in Step S164 on the display unit 94 and then ends the image generation process.

Next, the operation of the radiography apparatus 16 according to this embodiment will be described.

As described above, the radiography apparatus 16 according to this embodiment captures the first radiographic image, using the first radiation detector 20A, and captures the second radiographic image, using the second radiation detector 20B, in response to the imaging start command received from the console 18. First, the entire flow of a radiographic image capture process performed by the radiography apparatus 16 will be described.

When the imaging start command is received, the control unit 58A and the control unit 58B direct the first radiation detector 20A and the second radiation detector 20B to perform a reset operation, respectively. Even in a state in which the first radiation detector 20A and the second radiation detector 20B are not irradiated with the radiation R, charge is accumulated in the pixels 32 by a dark current. For this reason, a reset operation for sweeping the accumulated charge is performed. The reset operation that is performed in the first radiation detector 20A according to this embodiment is an example of a first reset operation according to the invention and the reset operation that is performed in the second radiation detector 20B is an example of a second reset operation according to the invention.

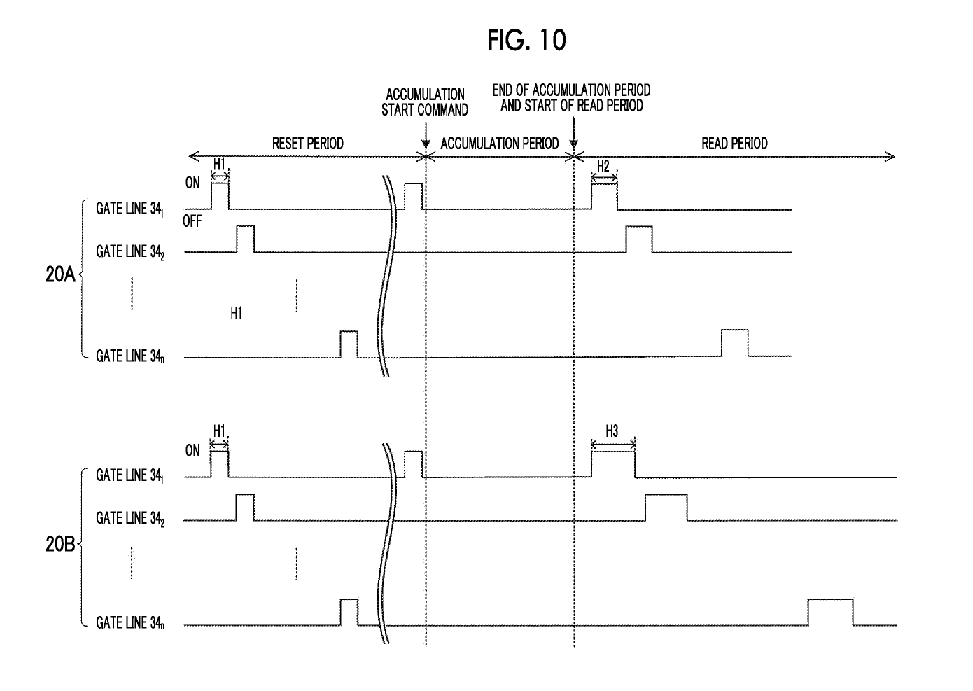

In this embodiment, for example, as illustrated in FIG. 10, in a reset period, the control unit 58A controls the gate line driver 52A such that the gate line driver 52A sequentially outputs an on signal to each gate line 34 of the first radiation detector 20A from a gate line 34₁ for a predetermined period H1. In addition, in the reset period, the control unit 58B controls the gate line driver 52B such that the gate line driver 52B sequentially outputs an on signal to each gate line 34 of the second radiation detector 20B from a gate line 34₁ for the predetermined period H1. FIG. 10 illustrates a case in which each of the first radiation detector 20A and the second radiation detector 20B includes n gate lines 34.

In the first radiation detector 20A, an electric signal, of which the level increases as the amount of charge output from the pixel 32 increases, is output to the integrated control unit 71 by the reset operation. The integrated control unit 71 detects the time when the emission of the radiation R has started, using the electric signal output by the reset operation. When the time when the emission of the radiation R has started is detected, the integrated control unit 71 outputs an accumulation start command to start a charge accumulation operation for generating a radiographic image to the control unit 58A and the control unit 58B.

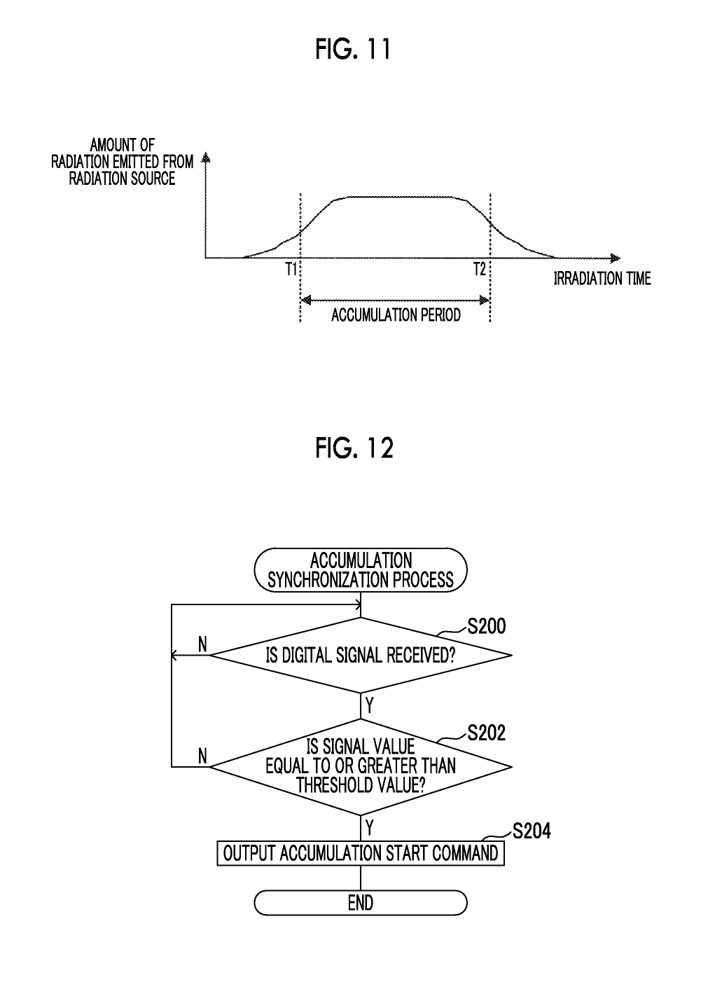

The time when the emission of the radiation R has started according to this embodiment is an example of a time related to the emission of radiation according to the invention. As illustrated in FIG. 11, the amount of radiation R emitted from the radiation source 14 of the radiation emitting apparatus 12 varies depending on the irradiation time. In the radiography apparatus 16 according to this embodiment, the period from a time T1 to a time T2 illustrated in FIG. 11, which depends on the amount of radiation R that is emitted from the radiation source 14 to the radiography apparatus 16, is an accumulation period which will be described below. Therefore, the time T1 is detected as the time when the emission of the radiation R has started. The time when the radiation source 14 actually starts the emission of the radiation R is different from the time when the radiography apparatus 16 starts to be irradiated with the radiation R. The time T1 is determined in terms of, for example, an error in the detection of the time.

For example, as illustrated in FIG. 10, when the accumulation start command is input, the control unit 58A ends the reset operation and performs the accumulation operation for the accumulation period. Specifically, the control unit 58A controls the gate line driver 52A such that an off signal is output from the gate line driver 52A to each gate line 34 of the first radiation detector 20A. Then, all of the thin film transistors 32C of the pixels 32 of the first radiation detector 20A are turned off. Similarly, when the accumulation start command is input, the control unit 58B ends the reset operation and controls the gate line driver 52B such that an off signal is output from the gate line driver 52B to each gate line 34 of the second radiation detector 20B for the accumulation period. Then, all of the thin film transistors 32C of the pixels 32 of the second radiation detector 20B are turned off.

When the accumulation period elapses, for example, as illustrated in FIG. 10, for a read period, the control unit 58A directs the gate line driver 52A to sequentially output the on signal to each gate line 34 of the first radiation detector 20A from the gate line 34₁ for a predetermined period H2 which is a read time per pixel. Similarly, when the accumulation period elapses, for the read period, the control unit 58B directs the gate line driver 52B to sequentially output the on signal to each gate line 34 of the second radiation detector 20B from the gate line 34₁ for a predetermined period H3 which is a read time per pixel.

In this embodiment, the predetermined periods H2 and H3 for which the on signal is output to the gate line 34 in the read period are longer than the predetermined period H1 for which the on signal is output to the gate line 34 in the reset period of each of the first radiation detector 20A and the second radiation detector 20B, which will be described in detail below. In addition, the predetermined period H3 for which the on signal is output to the gate line 34 of the second radiation detector 20B in the read period is longer than the predetermined period H2 for which the on signal is output to the gate line 34 of the first radiation detector 20A in the reset period of each of the first radiation detector 20A and the second radiation detector 20B.

In the radiography apparatus 16 according to this embodiment, the signal processing unit 54A and the signal processing unit 54B generate the first radiographic image data and the second radiographic image data, using the electric signals output from each pixel 32 for the read period, respectively.

Next, the operation of each of the integrated control unit 71, the control unit 58A, and the control unit 58B will be described in detail. FIG. 12 is a flowchart illustrating an example of the flow of an accumulation synchronization process performed by the integrated control unit 71. Specifically, when the imaging start command is received from the console 18, the CPU 72 of the integrated control unit 71 executes an accumulation synchronization processing program that is stored in the ROM of the memory 74 in advance to perform the accumulation synchronization process illustrated in FIG. 12. The accumulation synchronization processing program is an example of a program including a radiography program according to the invention.

In this embodiment, a case in which the integrated control unit 71 controls the start of a charge accumulation operation in the first radiation detector 20A and the second radiation detector 20B as an example of a charge accumulation operation control process to synchronize charge accumulation will be described.

In Step S200 of FIG. 12, the integrated control unit 71 determines whether the digital signal (hereinafter, referred to as a “reset digital signal”) obtained by converting the electric signal output from the pixel 32 of the first radiation detector 20A by the reset operation, using the signal processing unit 54A, has been received from the control unit 58A. In a case in which the reset digital signal has not been received, the determination result is “No” and the integrated control unit 71 waits until the reset digital signal is received. On the other hand, in a case in which the reset digital signal has been received, the determination result is “Yes” and the process proceeds to Step S202.

In Step S202, the integrated control unit 71 determines whether the value of the reset digital signal received in Step S200 is equal to or greater than a predetermined threshold value for detecting the start of the emission of the radiation R. In a case in which the value of the reset digital signal is less than the threshold value, the determination result is “No” and the process returns to Step S200. On the other hand, in a case in which the value of the reset digital signal is equal to or greater than the threshold value, the determination result is “Yes” and the process proceeds to Step S204. As such, the integrated control unit 71 according to this embodiment uses the method that detects the time when the reset digital signal is equal to or greater than the threshold value as the time when the emission of the radiation R has started. However, a method for detecting the time when the emission of the radiation R has started is not limited thereto. For example, the time when the reset digital signal is greater than the threshold value may be detected as the time when the emission of the radiation R has started or the time when a variation in the reset digital signal per unit time is equal to or greater than a predetermined threshold value may be detected as the time when the emission of the radiation R has started.

In Step S204, the integrated control unit 71 outputs an accumulation start command to the control unit 58A and the control unit 58B and ends the accumulation synchronization process.

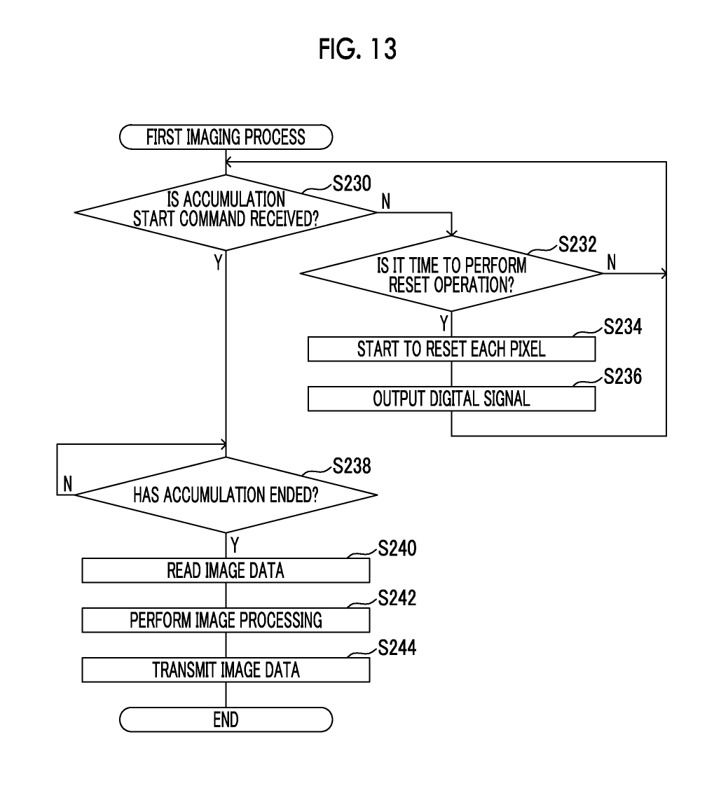

FIG. 13 is a flowchart illustrating an example of the flow of a first imaging process performed by the control unit 58A of the radiography apparatus 16. Specifically, when the imaging start command is received from the console 18, the CPU 60 of the control unit 58A executes a first imaging processing program that is stored in the ROM of the memory 62 in advance to perform the first imaging process illustrated in FIG. 13.

In Step S230 of FIG. 13, the control unit 58A determines whether a charge accumulation start command has been received from the integrated control unit 71. In a case in which the accumulation start command has not been received, the determination result is “No” and the process proceeds to Step S232.

In Step S232, the control unit 58A determines whether it is time to perform the reset operation. The time when the reset operation is performed is not particularly limited. For example, the reset operation may be performed whenever a predetermined period of time has elapsed since the imaging start command has been received from the console 18. In a case in which it is not time to perform the reset operation, the determination result is “No” and the process returns to Step S230. On the other hand, in a case in which it is time to perform the reset operation, the determination result is “Yes” and the process proceeds to Step S234.

In Step S234, the control unit 58A starts the reset operation. The electric signal generated by the charge flowing to each data line 36 in the reset operation is input to the signal processing unit 54A, is amplified by the variable gain pre-amplifier 82, and is converted into the reset digital signal by the A/D converter 88. The reset digital signal is input to the control unit 58A through the image memory 56A.

Then, in Step S236, the control unit 58A outputs the input reset digital signal to the integrated control unit 71 and the process returns to Step S230.

As described above, the reset digital signal that is output from the control unit 58A to the integrated control unit 71 by the above-mentioned reset operation is used to detect the start of the emission of the radiation R. Here, the reset digital signal that is generated by the charge output from all of the pixels 32 of the first radiation detector 20A may be output from the control unit 58A to the integrated control unit 71 or the reset digital signal that is generated by the charge output from the pixels 32 corresponding to at least one of the gate line 34 or the data line 36 predetermined to detect the start of the emission of the radiation R may be output.

In a case in which the accumulation start command has been received in Step S230, the determination result is “Yes” and the process proceeds to Step S238. In a case in which the accumulation start command has been received even though the on signal has not been output to the gate line 34ₙ in the reset operation started in Step S234, the control unit 58A ends the reset operation, proceeds from the reset period to the accumulation period, and turns off all of the thin film transistors 32C of the pixels 32 of the first radiation detector 20A.

FIG. 10 illustrates a case in which the reset operation is performed for the pixels 32 including the thin film transistors 32C controlled by the control signal flowing through the gate line 34₁ and then the accumulation start command is received. In this case, the on signal is not output to the gate line 34₂ and the subsequent gate lines 34.

In Step S238, the control unit 58A determines whether to end the accumulation of charge. A method for determining whether to end the accumulation of charge is not particularly limited. For example, in a case in which a predetermined accumulation period has elapsed since the accumulation start command has been received, the control unit 58A may determine to end the accumulation of charge. In a case in which the predetermined accumulation period has not elapsed, the determination result is “No” and the control unit 58A waits until the predetermined accumulation period elapses. On the other hand, in a case in which the predetermined accumulation period has elapsed, the determination result is “Yes” and the process proceeds to Step S240.

In Step S240, the control unit 58A proceeds from the accumulation period to the read period and controls the gate line driver 52A such that the gate line driver 52A sequentially outputs the on signal to each gate line 34 of the first radiation detector 20A for the predetermined period H2. Then, the lines of the thin film transistors 32C connected to each gate line 34 are sequentially turned on and charge accumulated in each line of the capacitors 32B flows as an electric signal to each data line 36. Then, the electric signal that has flowed to each data line 36 is converted into digital image data by the signal processing unit 54A and is then stored in the image memory 56A.

For the read period, the charge that has been generated by irradiation with the radiation R and then accumulated is output from the pixel 32. For the reset period, the charge generated by, for example, a dark current in a state in which the radiation R is not emitted is output from the pixel 32. Therefore, the amount of charge output from the pixel 32 for the read period is more than that for the reset period. For this reason, in this embodiment, as illustrated in FIG. 10, the predetermined period H2 in the read period is longer than the predetermined period H1 in the reset period. It is preferable that the time required for the reset operation is short. Therefore, it is preferable that the predetermined period H1 is short.

Then, in Step S242, the control unit 58A performs image processing including various correction processes, such as offset correction and gain correction, for the image data stored in the image memory 56A in Step S240. Then, in Step S244, the control unit 58A transmits the image data (first radiographic image data) processed in Step S242 to the integrated control unit 71 and ends the first imaging process.

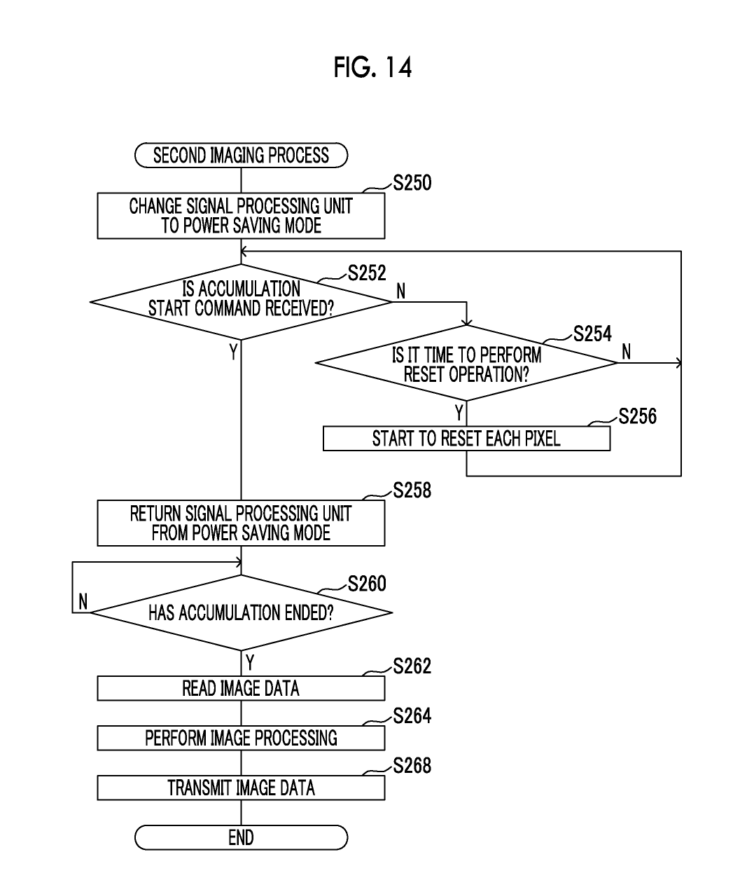

FIG. 14 is a flowchart illustrating an example of the flow of a second imaging process performed by the control unit 58B of the radiography apparatus 16. Specifically, when the imaging start command is received from the console 18, the CPU 60 of the control unit 58B executes a second imaging processing program that is stored in the ROM of the memory 62 in advance to perform the second imaging process illustrated in FIG. 14.

In Step S250 of FIG. 14, the control unit 58B suppresses power supplied from the power supply unit 70 to the signal processing unit 54B to change the signal processing unit 54B to a power saving mode. In the power saving mode, power that is supplied to the entire signal processing unit 54B may be suppressed or power that is supplied to each unit (see FIG. 4) of the signal processing unit 54B may be suppressed. Since the A/D converter 88 consumes a large amount of power, it is preferable to stop the driving of the A/D converter 88.

In the power saving mode according to this embodiment, the control unit 58B suppresses power supplied to the signal processing unit 54B. However, the invention is not limited thereto. For example, the control unit 58B may output a control signal for controlling the driving of each unit of the signal processing unit 54B and some or all of the units of the signal processing unit 54B may stop their driving or may be driven at a low speed in response to the control signal. As a result, the power supplied may be suppressed.

In this embodiment, the case in which the signal processing unit 54B is changed to the power saving mode has been described. However, the invention is not limited thereto. For example, each unit, such as the image memory 56B, that does not need to be driven in the reset operation and is considered not to have an effect on the generation of a radiographic image may be changed to the power saving mode.

Then, in Step S252, the control unit 58B determines whether the charge accumulation start command has been received from the integrated control unit 71. In case in which the accumulation start command has not been received, the determination result is “No” and the process proceeds to Step S254.

In Step S254, the control unit 58B determines whether it is time to perform the reset operation. The time when the reset operation is performed is not particularly limited. For example, the reset operation may be performed whenever a predetermined period of time has elapsed since the imaging start command has been received from the console 18. The reset operation in the first radiation detector 20A may be asynchronous with the reset operation in the second radiation detector 20B. In a case in which it is not time to perform the reset operation, the determination result is “No” and the process returns to Step S250. On the other hand, in a case in which it is time to perform the reset operation, the determination result is “Yes” and the process proceeds to Step S256.

In Step S256, the control unit 58B starts the above-mentioned reset operation and returns to Step S250. In the second radiation detector 20B, the electric signal generated by the charge that has flowed to each data line 36 in the reset operation is swept, without being converted into the reset digital signal, since the signal processing unit 54B is in the power saving mode. Therefore, the reset digital signal is not output from the control unit 58B to the integrated control unit 71.

In a case in which the accumulation start command has been received in Step S252, the determination result is “Yes” and the process proceeds to Step S258. In a case in which the accumulation start command has been received even though the on signal has not yet been output to the gate line 34ₙ in the reset operation started in Step S256, the control unit 58B ends the reset operation, proceeds from the reset period to the accumulation period, and turns off all of the thin film transistors 32C of the pixels 32 of the second radiation detector 20B.

Then, in Step S258, the control unit 58B stops the suppression of the supply of the power from the power supply unit 70 to the signal processing unit 54B and returns the signal processing unit 54B from the power saving mode.

Then, in Step S260, the control unit 58B determines whether to end the accumulation of charge. A method for determining whether to end the accumulation of charge is not particularly limited. For example, in a case in which a predetermined accumulation period has elapsed since the accumulation start command has been received, the control unit 58B may determine to end the accumulation of charge. In a case in which the predetermined accumulation period has not elapsed, the determination result is “No” and the control unit 58B waits until the predetermined accumulation period elapses. On the other hand, in a case in which the predetermined accumulation period has elapsed, the determination result is “Yes” and the process proceeds to Step S262.

Then, in Step S262, the control unit 58B controls the gate line driver 52B such that the gate line driver 52B sequentially outputs the on signal to each gate line 34 of the second radiation detector 20B for the predetermined period H3. Then, the lines of the thin film transistors 32C connected to each gate line 34 are sequentially turned on and charge accumulated in each line of the capacitors 32B flows as an electric signal to each data line 36. Then, the electric signal that has flowed to each data line 36 is converted into digital image data by the signal processing unit 54B and is then stored in the image memory 56B.

As described above, the amount of charge generated in each pixel 32 of the second radiation detector 20B is less than the amount of charge generated in each corresponding pixel 32 of the first radiation detector 20A. Therefore, in the radiography apparatus 16 according to this embodiment, so-called oversampling in which a read time per pixel for which the charge accumulated in the pixel 32 of the second radiation detector 20B is read is longer than that in the first radiation detector 20A. In this embodiment, for example, as illustrated in FIG. 10, the predetermined period H3 is longer than the predetermined period H2 in the first radiation detector 20A.

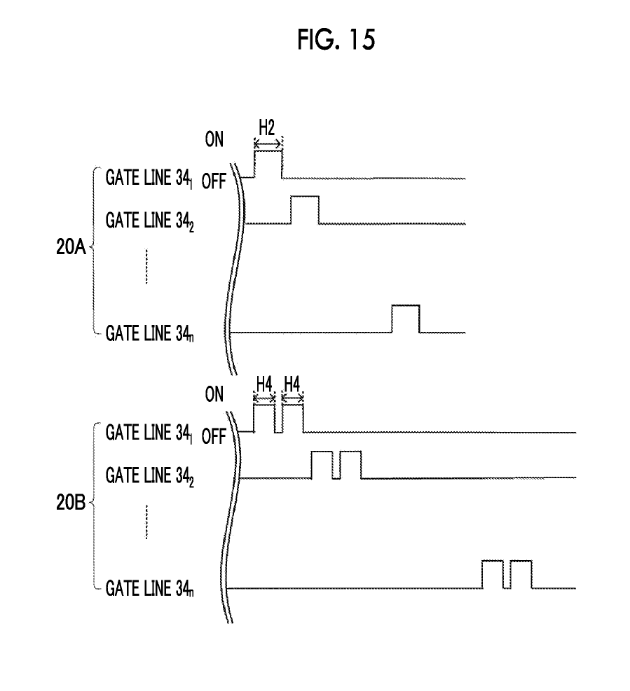

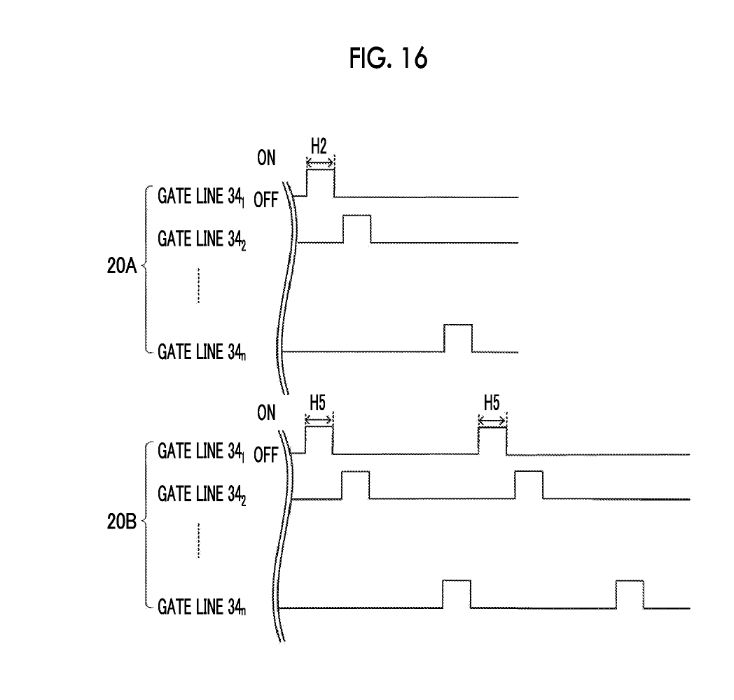

A method for performing the oversampling is not limited to the method illustrated in FIG. 10. For example, as illustrated in FIG. 15, the control unit 58B may continuously output the on signal to each gate line 34 for a predetermined period H4 a plurality of times (two times in FIG. 15) to perform the oversampling. In this case, the predetermined period H2 and the predetermined period H4 may be the same or different from each other. In addition, for example, as illustrated in FIG. 16, the control unit 58B may repeatedly perform a process that sequentially outputs the on signal to all of the gate lines 34 from the gate line 34₁ to the gate line 34ₙ for a predetermined period H5 and sequentially outputs the on signal to the gate lines 34₁ for the predetermined period H5 again. In this case, the predetermined period H2 and the predetermined period H5 may be the same or different from each other.

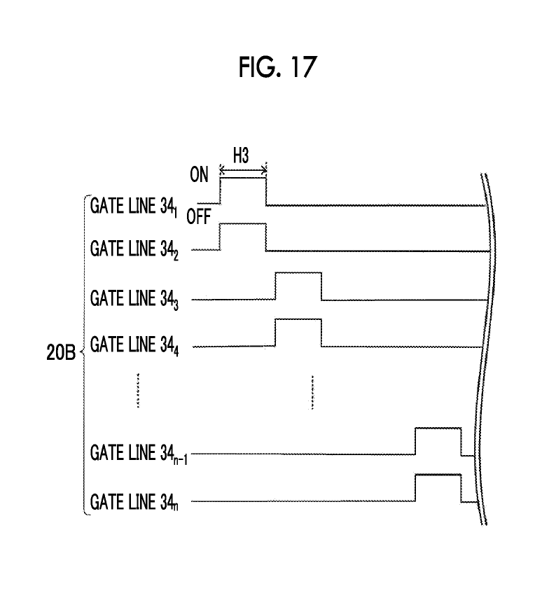

In this embodiment, the case in which the lines of the thin film transistors 32C connected to each gate line 34 are sequentially turned on and charge accumulated in each line of the capacitors 32B flows as an electric signal to each data line 36 has been described. However, a method for reading charge from the pixels 32 of the second radiation detector 20B (for outputting the electric signal) is not limited thereto. For example, since the amount of charge generated in each pixel 32 of the second radiation detector 20B is less than the amount of charge generated in each corresponding pixel 32 of the first radiation detector 20A, charge may be collectively read from a plurality of adjacent pixels 32 of the second radiation detector 20B. For example, as illustrated in FIG. 17, charge may be collectively read from the pixels 32 connected to each group of a plurality of adjacent gate lines 34. For example, FIG. 17 illustrates a case in which every two lines of the thin film transistors 32C connected to each gate line 34 are sequentially turned on and charge accumulated in every two lines of the capacitors 32B sequentially flows as an electric signal to each data line 36.

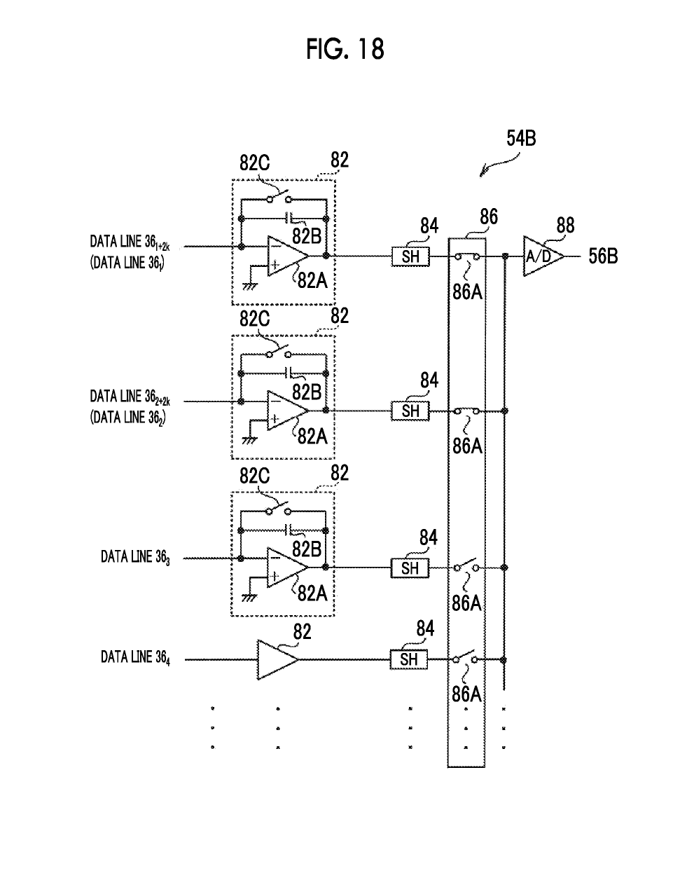

For example, as illustrated in FIG. 18, charge may be collectively read from the pixels 32 to each group of a plurality of adjacent data lines 36. For example, FIG. 18 illustrates a case in which m data lines 36 are provided, the sample-and-hold circuit 84 of the signal processing unit 54B samples the electric signals in every two data lines including a data line 36₁₊₂ₖ and a data line 36₂₊₂ₖ (k is an integer in the range of 0 to m/2) and the signals are selected by the switches 86A of the multiplexer 86 and are converted into digital signal by the A/D converter 88.

As such, in a case in which charge is collectively read from a plurality of adjacent pixels 32, for example, the quality of the generated second radiographic image, for example, the resolution of the generated second radiographic image is lower than that in a case in which charge is read from each pixel 32. However, as described above, in a case in which bone density is derived, bone density is preferably derived, not using the image indicated by DXA image data, but using the pixel value. Therefore, the influence of the reduction in image quality is small.

The amount of charge generated in each pixel 32 of the second radiation detector 20B is less than the amount of charge generated in each pixel 32 of the first radiation detector 20A and image quality is likely to be affected by noise. Therefore, the control unit 58B may adjust the gain of the variable gain pre-amplifier 82 of the signal processing unit 54B to reduce the influence of noise. In general, noise is generated due to a dark current in both stages before and behind the variable gain pre-amplifier 82 and the influence of noise caused by the radiation R overlaps noise generated in the stage before the variable gain pre-amplifier 82. Therefore, the gain of the variable gain pre-amplifier 82 is adjusted to adjust the ratio of noise generated in the stage before the variable gain pre-amplifier 82 and noise generated in the stage behind the variable gain pre-amplifier 82. As a result, it is possible to adjust the influence of noise in the stages before and behind the variable gain pre-amplifier 82. For example, when the gain of the variable gain pre-amplifier 82 increases, the influence of noise generated in the stage behind the variable gain pre-amplifier 82 is reduced. In addition, it goes without saying that the gain is adjusted in the range in which the capacitor 82B of the variable gain pre-amplifier 82 is not saturated.

Then, in Step S264, the control unit 58B performs image processing including various correction processes, such as offset correction and gain correction, for the image data stored in the image memory 56B in Step S262. Then, in Step S268, the control unit 58B transmits the image data (second radiographic image data) processed in Step S264 to the integrated control unit 71 and ends the second imaging process.

As described above, the radiography system 10 according to this embodiment includes: the radiography apparatus 16 including the first radiation detector 20A in which a plurality of pixels 32, each of which includes the sensor unit 32A that generates a larger amount of charge as it is irradiated with a larger amount of radiation R, are two-dimensionally arranged and the second radiation detector 20B which is provided so as to be stacked on the side of the first radiation detector 20A from which the radiation R is transmitted and emitted and in which a plurality of pixels 32, each of which includes the sensor unit 32A that generates a larger amount of charge as it is irradiated with a larger amount of radiation R, are two-dimensionally arranged; and the integrated control unit 71 that controls a charge accumulation operation in the plurality of pixels 32 of the first radiation detector 20A and a charge accumulation operation in the plurality of pixels 32 of the second radiation detector 20B, on the basis of the detection result of the time related to the emission of the radiation R using an electric signal which is obtained by converting charge generated in the pixels 32 of the first radiation detector 20A and of which the level increases as the amount of charge generated increases.

In the radiography apparatus 16 according to this embodiment, the amount of radiation that reaches the second radiation detector 20B is less than the amount of radiation that reaches the first radiation detector 20A. Therefore, the detection results of the time related to the emission of radiation in the first radiation detector 20A and the second radiation detector 20B are different from each other and the accumulation of charge in each pixel 32 of each of the radiation detectors is likely to be asynchronous. For this reason, in the radiography apparatus 16 according to this embodiment, in a case in which the time when the emission of the radiation R starts is detected by the electric signal output from the pixel 32 of the first radiation detector 20A, the accumulation start command is output to the first radiation detector 20A and the second radiation detector 20B to control the accumulation operation in the first radiation detector 20A and the second radiation detector 20B.

Therefore, according to the radiography system 10 according to each of the above-described embodiments, even when the amount of radiation R emitted to the second radiation detector 20B is less than the amount of radiation R emitted to the first radiation detector 20A, it is possible to synchronize the accumulation of charge.

In this embodiment, since the electric signal output from the pixel 32 of the second radiation detector 20B is not used to detect the start of the emission of radiation, it is possible to suppress power supplied from the power supply unit 70 for the reset period and to change the signal processing unit 54B to the power saving mode. Therefore, according to the radiography apparatus 16 according to this embodiment, it is possible to reduce power consumption. In particular, since the driving of the A/D converter 88 with a large amount of power consumption is stopped, it is possible to further reduce power consumption. In a case in which the A/D converter 88 is driven, power consumption increases and the amount of heat generated increases, which results in an increase in temperature around the A/D converter 88. Therefore, there is a concern that noise will be generated. However, in this embodiment, since the driving of the A/D converter 88 is stopped, it is possible to suppress the generation of noise caused by an increase in temperature.

In this embodiment, the case in which the integrated control unit 71 detects the time when the emission of the radiation R starts as the time related to the emission of the radiation R has been described. However, the invention is not limited thereto. For example, the integrated control unit 71 may detect the time when the emission of the radiation R is stopped like the time T2 illustrated in FIG. 11. In this case, for example, the integrated control unit 71 compares the value of the reset digital signal with a predetermined threshold value for detecting the stop of the emission of the radiation R. In a case in which the value of the reset digital signal is less than the threshold value, the integrated control unit 71 may determine that it is time to stop the emission of the radiation R. In addition, in a case in which the time when the emission of the radiation R is stopped is detected in this way, the integrated control unit 71 may output a command to end the charge accumulation operation to the control unit 58A and the control unit 58B. In this case, in a case in which the command is input, the control unit 58A and the control unit 58B end the accumulation period and proceed to the read period. Therefore, it is possible to synchronize the end of the accumulation period.

In this embodiment, the case in which an indirect-conversion-type radiation detector that converts radiation into light and converts the converted light into charge is applied to both the first radiation detector 20A and the second radiation detector 20B has been described. However, the invention is not limited thereto. For example, a direct-conversion-type radiation detector that directly converts radiation into charge may be applied to at least one of the first radiation detector 20A or the second radiation detector 20B.

In the radiography apparatus 16 according to this embodiment, the aspect in which the reset digital signal output from the signal processing unit 54A in the reset operation is used as the electric signal output from the pixel 32 of the first radiation detector 20A has been described. However, the electric signal used to detect the time related to the emission of the radiation R is not limited thereto. For example, a radiation detection pixel 32 including a thin film transistor 32C in which a source and a drain are short-circuited may be provided in the first radiation detector 20A and an electric signal generated by charge output from the radiation detection pixel 32 may be used.

In this embodiment, the aspect in which, in the second radiation detector 20B, every two lines of the thin film transistors 32C connected to each gate line 34 are sequentially turned on for the read period and charge accumulated in each line of the capacitors 32B sequentially flows as the electric signal to each data line 36 has been described. However, the invention is not limited thereto. For example, in both the first radiation detector 20A and the second radiation detector 20B, for the reset operation, as described with reference to FIGS. 17 and 18, charge may be collectively read from the pixels 32 connected to every group of a plurality of adjacent gate lines 34 or charge may be collectively read from the pixels 32 connected to every group of a plurality of adjacent data lines 36.

In this embodiment, the case in which the irradiation side sampling radiation detectors in which the radiation R is incident from the TFT substrates 30A and 30B are applied to the first radiation detector 20A and the second radiation detector 20B, respectively, has been described. However, the invention is not limited thereto. For example, a so-called penetration side sampling (PSS) radiation detector in which the radiation R is incident from the scintillator 22A or 22B may be applied to at least one of the first radiation detector 20A or the second radiation detector 20B.

In this embodiment, the case in which the radiography apparatus 16 is controlled by three control units (control units 58A, 58B, and 71) has been described. However, the invention is not limited thereto. For example, the control unit 58A may have the functions of the integrated control unit 71 or the radiography apparatus 16 may be controlled by one control unit.

In this embodiment, the case in which bone density is derived using the first radiographic image and the second radiographic image has been described. However, the invention is not limited thereto. For example, bone mineral content or both bone density and bone mineral content may be derived using the first radiographic image and the second radiographic image.

In this embodiment, the aspect in which the overall imaging processing program is stored (installed) in the ROM 90B in advance, the accumulation synchronization processing program is stored in the memory 74 in advance, the first imaging processing program is stored in the memory 62 in advance, and the second imaging processing program is stored in the memory 62 in advance has been described. However, the invention is not limited thereto. Each of the overall imaging processing program, the accumulation synchronization process program, the first imaging processing program, and the second imaging processing program may be recorded in a recording medium, such as a compact disk read only memory (CD-ROM), a digital versatile disk read only memory (DVD-ROM), or a universal serial bus (USB) memory, and then provided. In addition, each of the overall imaging processing program, the accumulation synchronization process program, the first imaging processing program, and the second imaging processing program may be downloaded from an external apparatus through a network.

In the radiography system according to the above-mentioned aspect of the present disclosure, the control unit may detect a start of emission of the radiation as the time related to the emission of the radiation.

In the radiography system according to the above-mentioned aspect of the present disclosure, the controller may detect a time when the electric signal becomes equal to or greater than a predetermined threshold as the start of the emission of the radiation.

In the radiography system according to the above-mentioned aspect of the present disclosure, the controller may detect a time when a variation in the electric signal per unit time becomes equal to or greater than a predetermined threshold as the start of the emission of the radiation.

In the radiography system according to the above-mentioned aspect of the present disclosure, the control unit may further perform control such that a first reset operation which resets the charge accumulated in the first plural pixels and a second reset operation which resets the charge accumulated in the first plural pixels are performed at a predetermined time before the emission of the radiation starts.

In the radiography system according to the above-mentioned aspect of the present disclosure, the first reset operation and the second reset operation may collectively reset at least one of the charge in each pixel in a plurality of adjacent rows or the charge in each pixel in a plurality of adjacent columns.

In the radiography system according to the above-mentioned aspect of the present disclosure, each of the first radiation detector and the second radiation detector may further include a signal processing unit that includes an amplifier to which the charge accumulated in the plural pixels is input as the electric signal and which amplifies the input electric signal, a sample-and-hold circuit that holds the electric signal amplified by the amplifier, and an analog/digital converter that converts the electric signal output from the sample-and-hold circuit into a digital signal, and performs a process of generating image data of a radiographic image from the input electric signal. A gain of the amplifier in the second radiation detector may be higher than a gain of the amplifier in the first radiation detector.

In the radiography system according to the above-mentioned aspect of the present disclosure, the second radiation detector may further include: a signal processing unit to which the charge accumulated in the first plural pixels is input as the electric signal and which performs a process of generating image data of a radiographic image from the electric signal; and a power control unit that controls the supply of power from a power supply unit which supplies power for driving the second radiation detector. The power control unit may suppress the supply of power from the power supply unit to the signal processing unit until the second radiation detector starts the accumulation of charge in the first plural pixels under the control of the control unit.

In the radiography system according to the above-mentioned aspect of the present disclosure, the signal processing unit may include an amplifier that amplifies the input electric signal, a sample-and-hold circuit that holds the electric signal amplified by the amplifier, and an analog/digital converter that converts the electric signal output from the sample-and-hold circuit into a digital signal. The power control unit may perform control such that the supply of power from the power supply unit to the analog digital converter is suppressed.

In the radiography system according to the above-mentioned aspect of the present disclosure, after controlling the charge accumulation operation, the control unit may perform a control operation that reads the charge accumulated in the first plural pixels and a control operation that sets a read time per pixel in the second radiation detector to be longer than a read time per pixel in the first radiation detector and reads the charge accumulated in the first plural pixels.

In the radiography system according to the above-mentioned aspect of the present disclosure, the control unit may collectively read at least one of the charge accumulated in each pixel in a plurality of adjacent rows or the charge accumulated in each pixel in a plurality of adjacent columns.

In the radiography system according to the above-mentioned aspect of the present disclosure, the control unit may control at least one of the start of the charge accumulation operation or the end of the charge accumulation operation as a control process for the charge accumulation operation.

In the radiography system according to the above-mentioned aspect of the present disclosure, each of the first radiation detector and the second radiation detector may include a light emitting layer that is irradiated with radiation and emits light. The plural pixels of each of the first radiation detector and the second radiation detector may receive the light, generate the charge, and accumulate the charge. The light emitting layer of the first radiation detector and the light emitting layer of the second radiation detector may have different compositions.

In the radiography system according to the above-mentioned aspect of the present disclosure, the light emitting layer of the first radiation detector may include CsI and the light emitting layer of the second radiation detector may include GOS.

The radiography system according to the above-mentioned aspect of the present disclosure may further includes a derivation unit that derives at least one of bone mineral content or bone density, using a first radiographic image captured by the first radiation detector and a second radiographic image captured by the second radiation detector.

According to the present disclosure, it is possible to synchronize the accumulation of charge even when the amount of radiation emitted to the second radiation detector is less than the amount of radiation emitted to the first radiation detector.

What is claimed is:

1. A radiography system comprising:
a radiography apparatus comprising a first radiation detector in which a first plurality of pixels, each of which includes a conversion element that generates a larger amount of charge as it is irradiated with a larger amount of radiation, are two-dimensionally arranged, and a second radiation detector provided on a side of the first radiation detector from which the radiation is transmitted and emitted and in which a second plurality of pixels, each of which includes a conversion element that generates a larger amount of charge as it is irradiated with a larger amount of radiation, are two-dimensionally arranged; and
a controller that executes a process, the process comprising:
obtaining an electric signal, which is converted from charge generated in the first plurality of pixels and of which the level increases as the amount of charge increases;
detecting a time related to the emission of the radiation from the obtained electric signal; and
controlling a first charge accumulation operation in the first plurality of pixels and a second charge accumulation operation in the second plurality of pixels on the basis of the detected time.

2. The radiography system according to claim 1,
wherein the controller detects a start of emission of the radiation as the time related to the emission of the radiation.

3. The radiography system according to claim 2,
wherein the controller detects a time when the electric signal becomes equal to or greater than a predetermined threshold as the start of the emission of the radiation.

4. The radiography system according to claim 2,
wherein the controller detects a time when a variation in the electric signal per unit time becomes equal to or greater than a predetermined threshold as the start of the emission of the radiation.

5. The radiography system according to claim 1,
wherein the controller further performs control such that a first reset operation which resets the charge accumulated in the first plurality of pixels and a second reset operation which resets the charge accumulated in the second plurality of pixels are performed at a predetermined time before the emission of the radiation starts.

6. The radiography system according to claim 2,
wherein the controller further performs control such that a first reset operation which resets the charge accumulated in the first plurality of pixels and a second reset operation which resets the charge accumulated in the second plurality of pixels are performed at a predetermined time before the emission of the radiation starts.

7. The radiography system according to claim 5,
wherein the first reset operation and the second reset operation collectively reset at least one of the charge in each pixel in a plurality of adjacent rows or the charge in each pixel in a plurality of adjacent columns.

8. The radiography system according to claim 6,
wherein the first reset operation and the second reset operation collectively reset at least one of the charge in each pixel in a plurality of adjacent rows or the charge in each pixel in a plurality of adjacent columns.

9. The radiography system according to claim 1,
wherein each of the first radiation detector and the second radiation detector further comprises a signal processing unit that comprises an amplifier to which the charge accumulated in the plurality of pixels is input as the electric signal and which amplifies the input electric signal, a sample-and-hold circuit that holds the electric signal amplified by the amplifier, and an analog/digital converter that converts the electric signal output from the sample-and-hold circuit into a digital signal, and performs a process of generating image data of a radiographic image from the input electric signal, and
wherein a gain of the amplifier in the second radiation detector is higher than a gain of the amplifier in the first radiation detector.

10. The radiography system according to claim 1,
wherein the second radiation detector further comprises:
a signal processing unit to which the charge accumulated in the second plurality of pixels is input as the electric signal and which performs a process of generating image data of a radiographic image from the electric signal; and
a power controller that controls the supply of power from a power supply unit which supplies power for driving the second radiation detector, and
the power controller suppresses the supply of power from the power supply unit to the signal processing unit until the second radiation detector starts the accumulation of charge in the second plurality of pixels under the control of the controller.

11. The radiography system according to claim 10,
wherein the signal processing unit comprises an amplifier that amplifies the input electric signal, a sample-and-hold circuit that holds the electric signal amplified by the amplifier, and an analog/digital converter that converts the electric signal output from the sample-and-hold circuit into a digital signal, and
the power controller performs control such that the supply of power from the power supply unit to the analog digital converter is suppressed.

12. The radiography system according to claim 1,
wherein, after controlling the charge accumulation operation, the controller performs a control operation that reads the charge accumulated in the first plurality of pixels and a control operation that sets a read time per pixel in the second radiation detector to be longer than a read time per pixel in the first radiation detector and reads the charge accumulated in the second plurality of pixels.

13. The radiography system according to claim 1,
wherein the controller collectively reads at least one of the charge accumulated in each pixel in a plurality of adjacent rows or the charge accumulated in each pixel in a plurality of adjacent columns.

14. The radiography system according to claim 1,
wherein the controller controls at least one of the start of the charge accumulation operation or the end of the charge accumulation operation as a control process for the charge accumulation operation.

15. The radiography system according to claim 1,
wherein each of the first radiation detector and the second radiation detector comprises a light emitting layer that is irradiated with radiation and emits light,
the plurality of pixels of each of the first radiation detector and the second radiation detector receive the light, generate the charge, and accumulate the charge, and
the light emitting layer of the first radiation detector and the light emitting layer of the second radiation detector have different compositions.

16. The radiography system according to claim 15,
wherein the light emitting layer of the first radiation detector includes CsI, and
the light emitting layer of the second radiation detector includes GOS.

17. The radiography system according to claim 1, further comprising:
a derivation unit that derives at least one of bone mineral content or bone density, using a first radiographic image captured by the first radiation detector and a second radiographic image captured by the second radiation detector.

18. A radiography method that is performed by a radiography apparatus comprising a first radiation detector in which a first plurality of pixels, each of which includes a conversion element that generates a larger amount of charge as it is irradiated with a larger amount of radiation, are two-dimensionally arranged, and a second radiation detector which is provided on a side of the first radiation detector from which the radiation is transmitted and emitted and in which a second plurality of pixels, each of which includes a conversion element that generates a larger amount of charge as it is irradiated with a larger amount of radiation, are two-dimensionally arranged, the method comprising:
obtaining an electric signal, which is converted from charge generated in the first plurality of pixels and of which the level increases as the amount of charge increases;
detecting a time related to the emission of the radiation from the obtained electric signal; and
controlling a first charge accumulation operation in the first plurality of pixels and a second charge accumulation operation in the second plurality of pixels on the basis of the detected time.

19. A non-transitory computer readable storage medium storing a radiography program that causes a computer to execute a process of controlling a radiography apparatus, the radiography apparatus comprising a first radiation detector in which a first plurality of pixels, each of which includes a conversion element that generates a larger amount of charge as it is irradiated with a larger amount of radiation, are two-dimensionally arranged, and a second radiation detector which is provided on a side of the first radiation detector from which the radiation is transmitted and emitted and in which a second plurality of pixels, each of which includes a conversion element that generates a larger amount of charge as it is irradiated with a larger amount of radiation, are two-dimensionally arranged, and the process comprising:
obtaining an electric signal, which is converted from charge generated in the first plurality of pixels and of which the level increases as the amount of charge increases;
detecting a time related to the emission of the radiation from the obtained electric signal; and
controlling a first charge accumulation operation in the first plurality of pixels and a second charge accumulation operation in the second plurality of pixels on the basis of the detected time.
